(19) United States
(12) Patent Application Publication
(10) Pub. No.: US 2023/0190112 A1
(43) Pub. Date: Jun. 22, 2023

(54) LOW-COST AND SCALABLE SCREEN PRINTED WEARABLE HUMAN BODY TEMPERATURE SENSOR

(71) Applicant: Qatar Foundation for Education, Science and Community Development, Doha (QA)

(72) Inventors: Amine Bermak, Doha (QA); Shawkat Ali, Doha (QA); Saleem Khan, Doha (QA)

(21) Appl. No.: 17/900,261

(22) Filed: Aug. 31, 2022

Related U.S. Application Data
(60) Provisional application No. 63/238,897, filed on Aug. 31, 2021.

(51) Int. Cl. A61B 5/01; H01B 13/00; B33Y 80/00; B33Y 10/00; G01K 13/20

(52) U.S. Cl. CPC A61B 5/01; H01B 13/0026; B33Y 80/00; B33Y 10/00; G01K 13/20; A61B 2560/0247; A61B 2562/162; A61B 2562/125

(57) ABSTRACT

A wearable sensor for real-time human body temperature measurement is provided. The wearable sensor includes a substrate, a first electrode on the substrate, a second electrode on the substrate, the second electrode being spaced apart from the first electrode, and a sensing film on the substrate. The sensing film is electrically and/or spatially disposed between the first electrode and the second electrode. A resistance between the first electrode and the second electrode changes in response to a change in temperature surrounding the sensing film.

### PRIORITY CLAIM

The present application claims priority to and the benefit of U.S. Provisional Patent Application No. 63/238,897, filed on Aug. 31, 2021, the entirety of which is incorporated herein by reference.

### BACKGROUND

Human body temperature measurement is an important vital biosignal, which may be an early indication of a variety of illnesses. There are various types of body temperature measurement devices. Examples of body temperature measurement devices may include a mercury thermometer, an infrared thermometer, a bimetal type thermometer, and a thermocouple thermometer.

### SUMMARY

The disclosure relates to a low-cost and scalable wearable human body temperature sensor.

In light of the present disclosure, and without limiting the scope of the disclosure in any way, in an aspect of the present disclosure, which may be combined with any other aspect listed herein unless specified otherwise, a wearable sensor for real-time human body temperature measurement is provided. The wearable sensor includes a substrate, a first electrode on the substrate, a second electrode on the substrate, the second electrode being spaced apart from the first electrode, and a sensing film on the substrate. The sensing film is electrically and/or spatially disposed between the first electrode and the second electrode. A resistance between the first electrode and the second electrode changes in response to a change in temperature surrounding the sensing film.

In an aspect of the present disclosure, which may be combined with any other aspect listed herein unless specified otherwise, the wearable sensor further includes an encapsulation layer on the sensing film.

In an aspect of the present disclosure, which may be combined with any other aspect listed herein unless specified otherwise, the encapsulation layer covers the first electrode, the second electrode, and the sensing film to protect the first electrode, the second electrode, and the sensing film from an environmental factor.

In an aspect of the present disclosure, which may be combined with any other aspect listed herein unless specified otherwise, the environmental factor comprises at least one of humidity and oxidation.

In an aspect of the present disclosure, which may be combined with any other aspect listed herein unless specified otherwise, the encapsulation layer comprises polydimethylsiloxane.

In an aspect of the present disclosure, which may be combined with any other aspect listed herein unless specified otherwise, the encapsulation layer is waterproof.

In an aspect of the present disclosure, which may be combined with any other aspect listed herein unless specified otherwise, a detection temperature range of the wearable sensor is in a range of about 28° C. to about 50° C.

In an aspect of the present disclosure, which may be combined with any other aspect listed herein unless specified otherwise, the sensing film comprises carbon black.

In an aspect of the present disclosure, which may be combined with any other aspect listed herein unless specified otherwise, the first electrode and the second electrode comprise silver nano-particles.

In an aspect of the present disclosure, which may be combined with any other aspect listed herein unless specified otherwise, the first electrode and the second electrode comprise an interdigital electrode having a comb-shaped arrangement.

In an aspect of the present disclosure, which may be combined with any other aspect listed herein unless specified otherwise, the wearable sensor further comprises a detection/processing circuit configured to process the change of the resistance.

In an aspect of the present disclosure, which may be combined with any other aspect listed herein unless specified otherwise, the detection/processing circuit is configured to determine a human body temperature based on the change of the resistance.

In an aspect of the present disclosure, which may be combined with any other aspect listed herein unless specified otherwise, the detection/processing circuit is configured to determine a respiration rate based on the change of the resistance.

In an aspect of the present disclosure, which may be combined with any other aspect listed herein unless specified otherwise, the change of the resistance is reversible.

In light of the present disclosure, and without limiting the scope of the disclosure in any way, in an aspect of the present disclosure, which may be combined with any other aspect listed herein unless specified otherwise, a method of manufacturing a wearable sensor for real-time human body temperature measurement is provided. The method includes providing a substrate, printing a first electrode and a second electrode on the substrate, the second electrode being spaced apart from the first electrode, and printing a sensing film on the substrate. The sensing film is electrically and/or spatially disposed between the first electrode and the second electrode. A resistance between the first electrode and the second electrode changes in response to a change in temperature surrounding the sensing film.

In an aspect of the present disclosure, which may be combined with any other aspect listed herein unless specified otherwise, the method further comprises printing an encapsulation layer on the sensing film, the first electrode, and the second electrode to protect the sensing film, the first electrode, and the second electrode from an environmental factor.

In an aspect of the present disclosure, which may be combined with any other aspect listed herein unless specified otherwise, at least one of the first electrode, the second electrode, and the sensing film is printed using a screen-printing technique or a 3D printing technique.

The reader will appreciate the foregoing details, as well as others, upon considering the following detailed description of certain non-limiting embodiments including a low-cost and scalable wearable human body temperature sensor according to the present disclosure.

### BRIEF DESCRIPTION OF THE DRAWINGS

FIG. 1 is a diagram of a wearable sensor according to an example of the present disclosure.

FIG. 2 is a diagram of electrodes of the wearable sensor of FIG. 1.

FIG. 3 is an example of a cross-sectional view of the wearable sensor of FIG. 1 along the line of A-A.

FIG. 4 is a diagram of a wearable sensor and a detection/processing circuit according to an example of the present disclosure.

FIG. 5 is a diagram of a wearable sensor attached to a wrist of a user according to an example of the present disclosure.

FIG. 6 is a graph showing experimental results of the response/recovery time of a wearable sensor according to an example of the present disclosure.

FIG. 7 is a graph showing experimental results of a resistance test of a wearable sensor according to an example of the present disclosure.

FIG. 8 is a flowchart illustrating an example method for manufacturing a wearable sensor according to an example embodiment of the present disclosure.

### DETAILED DESCRIPTION

The present disclosure generally relates to a low-cost and scalable wearable human body temperature sensor.

Human body temperature is an important vital sign that can be used as an early indication for a variety of illnesses. Additionally, measuring the temperature of an exhaled breath can be used to diagnose several chronic diseases. Furthermore, there are situations where it is important to have constant body temperature measurement to ensure the health of an individual, such as during athletic competitions. However, present temperature sensors are either too bulky or too expensive to constantly measure body temperature or cannot easily measure the temperature of exhaled breath due to the sensor's size.

Aspects of the present disclosure may provide a low-cost body temperature sensor that is designed to monitor a user's temperature by either weaving the sensor into clothing or placing the sensor directly in contact with the user's skin. The sensor may include a polyimide (PI) substrate, silver nano-particles interdigital electrode (IDE), carbon black sensing film, PDMS encapsulation, and silvers connecting pads. The sensor may be created using screen printing on a biocompatible substrate, which may reduce cost and allows the sensor to be manufactured in a range of sizes.

In some examples, the sensor may be fully encapsulated, waterproof, and immune to environmental effects. The temperature sensor may include two terminals that may change the electrical resistance against temperature variation. Variation in resistance may be transformed into voltage levels with a readout circuit (a voltage divider network), and based on the voltage levels, the sensor may provide temperature measurements.

According to one or more embodiments, the disclosure provides a human body temperature sensor deployable on the human wrist. The sensor may be developed by using a low-cost screen printing technology on a biocompatible substrate and with a detection range between 28 to 50° C. The sensor can be used for various applications, such as human body skin temperature sensing, deep body temperature sensing, and respiration rate sensing. In some examples, the sensor may be resistive type and provide a variation in the terminal resistance against input entity. It can be easily interfaced with electronic processing circuits by using a simple voltage divider network, which may convert the resistance variation into voltage variation for the further process.

According to one or more embodiments, the sensor may include a PI substrate, silver IDE, and carbon black sensing film. The sensor may be encapsulated with PDMS to avoid environmental effects. In some examples, the sensor device may be connected to a readout module.

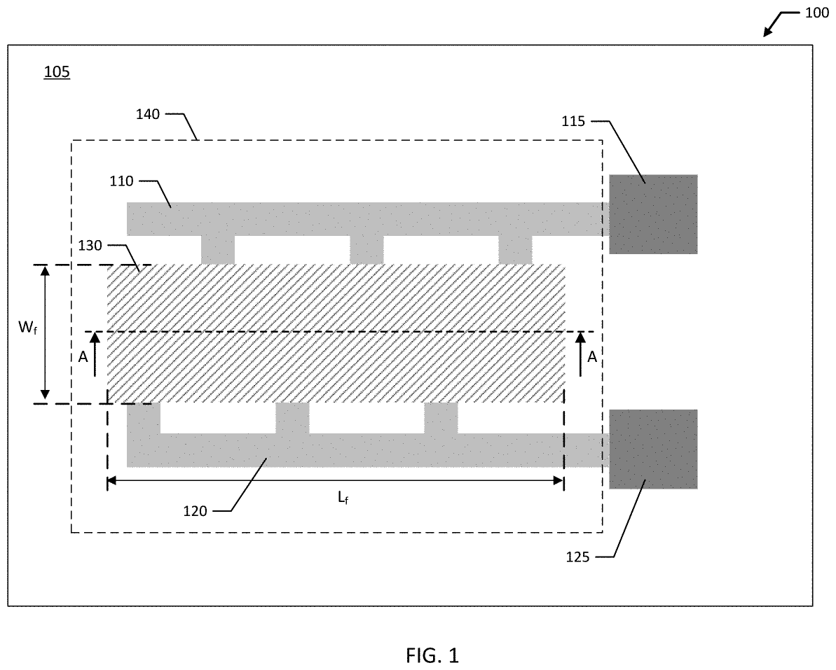

FIG. 1 illustrates an example wearable sensor 100 according to an example of the present disclosure. As illustrated in FIG. 1, the wearable sensor 100 may include a substrate 105, a first electrode 110, and a second electrode 120. The first electrode 110 and the second electrode 120 may be disposed on the substrate 105. The second electrode 120 may be spaced apart from the first electrode 110.

In some examples, the substrate 105 may be made with a thermally sensitive and/or biocompatible material. For example, in some examples, the substrate 105 may be made with polyimide. In other examples, the substrate 105 may be made with any other suitable material.

In some examples, at least one of the first electrode 110 and the second electrode 120 may be made with silver (e.g., silver nano-particles). In other examples, the first electrode 110 and the second electrode 120 may be made with any other suitable material (e.g., any other suitable conductive material, such as metal).

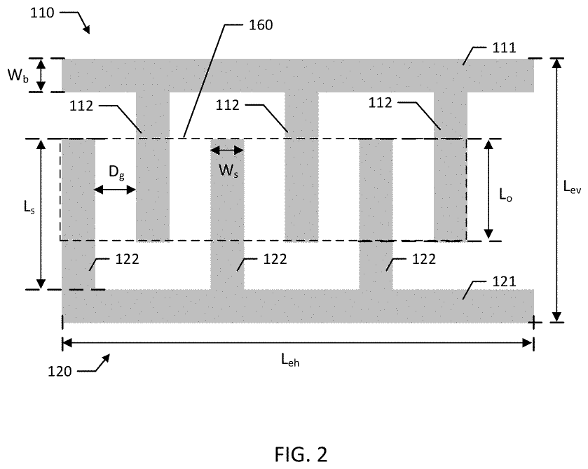

The first electrode 110 and the second electrode 120 are separately shown in FIG. 2. In some examples, the first electrode 110 and the second electrode 120 may be an interdigital electrode. For example, the first electrode 110 and the second electrode 120 may have a comb-shaped arrangement as shown in FIG. 2. The interdigital electrode may have a horizontal length Lₑₕ and/or a vertical length L_{ev [A1]} in a range of about 2 mm to about 6 mm. In other examples, the interdigital electrode may have any other suitable horizontal/vertical length.

The first electrode 110 may include a first base electrode 111, and one or more first sub-electrodes 112. The one or more first sub-electrodes 112 may protrude from the first base electrode 111. Similarly, the second electrode 120 may include a second base electrode 121, and one or more second sub-electrodes 122, and the one or more second sub-electrodes 122 may protrude from the second base electrode 121. A gap D_{g} may be formed between first sub-electrode 112 and the second sub-electrode 122. In some examples, the gap D_{g} may be in a range of about 0.1 mm to about 0.5 mm. In other examples, the gap D_{g} may have any other suitable distance.

In some examples, the first base electrode 111 and the second base electrode 121 may have a width W_{b} in a range of about 0.5 mm to about 1.5 mm. In other examples, the first base electrode 111 and the second base electrode 121 may have any other suitable width. In some examples, the first sub-electrodes 112 and the second sub-electrodes 122 may have a width Wₛ in a range of about 0.1 mm to about 0.5 mm. In other examples, the first base electrode 111 and the second base electrode 121 may have any other suitable width. In some examples, the first sub-electrodes 112 and the second sub-electrodes 122 may have a length Lₛ in a range of about 1 mm to about 3 mm. In other examples, the first base electrode 111 and the second base electrode 121 may have any other suitable length.

The wearable sensor 100 may further include a sensing film 130 disposed on the substrate 105. The sensing film 130 may be electrically and/or spatially disposed between the first electrode 110 and the second electrode 120. For example, the gap between the first electrode 110 and the second electrode 120 may be filled with the sensing film 130.

A resistance between the first electrode 110 and the second electrode 120 may change in response to a change in temperature surrounding the sensing film 130. For example, the electrical property (e.g., resistance value) of the sensing film 130 measured from the first electrode 110 and the second electrode 120 may change in response to the change in temperature surrounding the sensing film 130 (e.g., the change in temperature of a human body to which the wearable sensor 100 is attached). In some examples, the sensing film 130 may be made with carbon black. In other examples, the sensing film 130 may be made with any other suitable material.

In some examples, the sensing film 130 may have a length L_{f} in a range of about 2 mm to about 6 mm. In other examples, the sensing film 130 may have any other suitable length. In some examples, the sensing film 130 may have a width W_{f} in a range of about 1 mm to about 3 mm. In other examples, the sensing film 130 may have any other suitable width.

In some examples, the first sub-electrodes 112 and the second sub-electrodes 122 may overlap to each other (when viewed from a side of the wearable sensor 100), as shown in FIG. 2 (hereinafter, “overlapped portion 160”). In some examples, the overlapped portion 160 may have a length Lₒ in a range of about 0.5 mm to about 1.5 mm. In other examples, the overlapped portion 160 may have any other suitable length. In some examples, the sensing film 130 may cover at least the entire portion of the overlapped portion 160. In other examples, the sensing film 130 may cover only a portion of the overlapped portion 160.

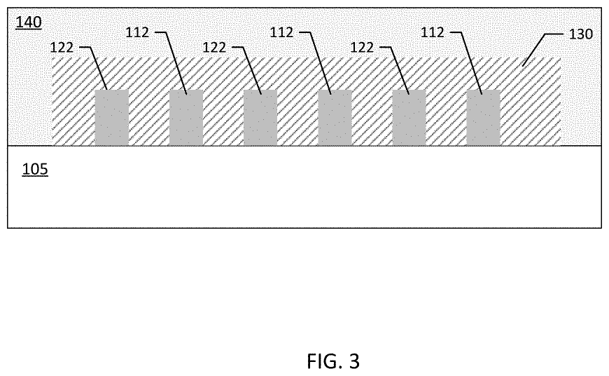

In some examples, the wearable sensor 100 may further include an encapsulation layer 140. The encapsulation layer 140 is shown as transparent in FIG. 1 for illustrative purpose only, and the encapsulation layer 140 may or may not be transparent. The encapsulation layer 140 may be disposed on the sensing film 130, as shown in FIGS. 1 and 3. In some examples, the encapsulation layer 140 may cover the first electrode 110, the second electrode 120, and the sensing film 130 to protect the first electrode 110, the second electrode 120, and the sensing film 130 from an environmental factor, such as humidity and/or oxidation. For example, the electrodes 110, 120 may be prone to oxidation and the sensing layer 130 may be sensitive to humidity.

In some examples, the encapsulation layer 140 may be made with polydimethylsiloxane. In other examples, the encapsulation layer 140 may be made with any other suitable material. In some examples, the encapsulation layer 140 may be waterproof.

In some examples, the wearable sensor 100 may further include a first connecting pad 115 electrically coupled to the first electrode 110, and a second connecting pad 125 electrically coupled to the second electrode 120. The first and second connecting pads 115, 125 may serve as a readout terminal and be used as a contact point with a detection/processing circuit when measuring a resistance between the first electrode 110 and the second electrode 120.

In some examples, at least one of the first connecting pad 115 and the second connecting pad 125 may be made with silver (e.g., silver nano-particles). In other examples, the first connecting pad 115 and the second connecting pad 125 may be made with any other suitable material (e.g., any other suitable conductive material, such as metal).

In some examples, at least one of the first electrode 110, the second electrode 120, the sensing film 130, the encapsulation layer 140, the first connecting pad 115, and the second connecting pad 125 may be printed using a screen-printing technique or a 3D printing technique. In other examples, the first electrode 110, the second electrode 120, the sensing film 130, the encapsulation layer 140, the first connecting pad 115, and the second connecting pad 125 may be printed using any other suitable process.

In some examples, the wearable sensor 100 can detect/measure a temperature in a range of about 28° C. to about 50° C., for example, about 28° C. to about 35° C., about 35° C. to about 40° C., about 40° C. to about 45° C., or about 45° C. to about 50° C. In other examples, the wearable sensor 100 can detect/measure any other suitable temperature.

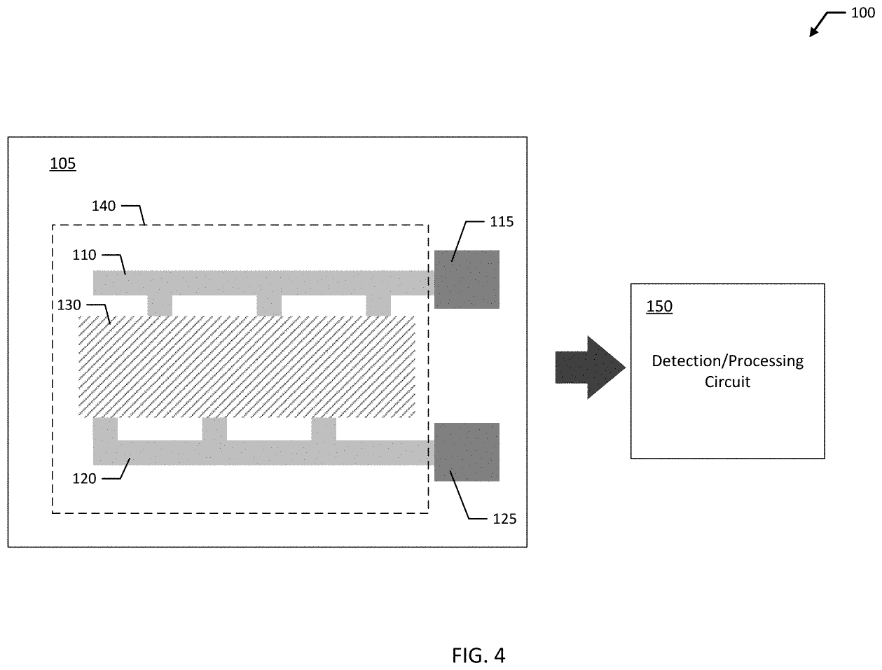

Referring to FIG. 4, in some examples, the wearable sensor 100 may be provided with a detection/processing circuit 150. The detection/processing circuit 150 may process the change of the resistance. In some examples, the detection/processing circuit 150 may determine a human body temperature (e.g., skin/deep body temperature) based on the change of the resistance. The detection/processing circuit 150 may transform the change in resistance into voltage levels. In some examples, the detection/processing circuit 150 may include a voltage divider network that may convert a resistance variation into a voltage variation for the further process.

In some examples, the detection/processing circuit 150 may determine a respiration rate based on the change of the resistance. For example, the wearable sensor 100 can be used in a facial mask (e.g., attached thereto), and the wearable sensor 100 may be interacting with inhale/exhale cycles and/or read the deep body temperature. The wearable sensor 100 may detect/measure the respiration rate of the user wearing the facial mask with the wearable sensor 100 (e.g., on the mouth or nose) based on the detected inhale/exhale cycles and/or change in temperature. For example, during the inhale and exhale of the breath, the temperature on the surface of the wearable sensor may change, which may cause a change in resistance of the wearable sensor. The change in resistance may provide information about the breathing rate (respiration rate) on the time scale. For a normal human, the respiration rate may be 12 to 20 breaths per second.

In this way, aspects of the present disclosure may provide a wearable sensor that is capable of monitoring the breathing of a user, as there might be some temperature variations during the inhale and exhale cycles. This breathing data can be used to continuously monitor the heath conditions of the user, and it may provide a deeper insight into the health conditions of a patient with chronic diseases.

In some examples, the change of the resistance of the wearable sensor may be reversible. That is, if the temperature surrounding the wearable sensor 100 returns back to its original temperature (e.g., T2 to T1), the wearable sensor 100 may not maintain the changed resistance value (e.g., R2 measured at T2) and/or also return back to its original resistance value (e.g., R1 measured at the original temperature T1).

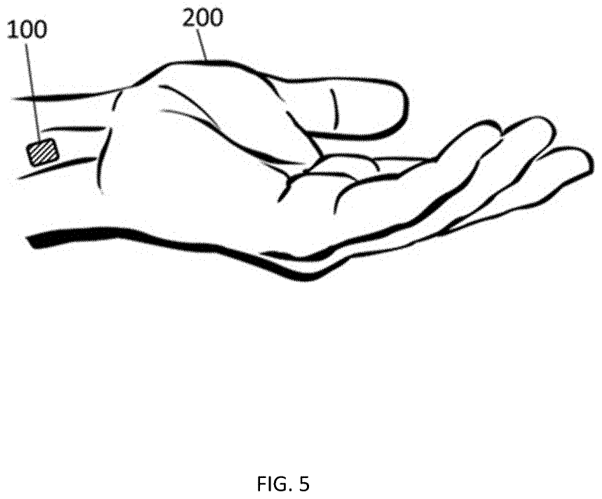

In some examples, the wearable sensor 100 can be scaled according to the requirement from micro size to centimeter size. The wearable sensor 100 can be placed on the human body, for example, on the wrist of the hand 200 of a user, as shown in FIG. 5, or any temperature-readable area of the user. The wearable sensor may be flexible, conformable, and can become an integral part of a wearable system. The wearable sensor can be shaped for applications, such as wrist band, facial mask, fabric, or any non-planar substrate that may interact with a human body for a temperature reading. The temperature measurement from the wearable sensor can be used for prolonged analysis of the human body skin and deep body temperature. The wearable sensor according to the present disclosure can be worn easily by a user, as the components of the wearable sensor (e.g., substrate, electrodes, sensing film, or encapsulation layer) may be flexible, foldable, and/or stretchable without compromising the overall performance of the sensor.

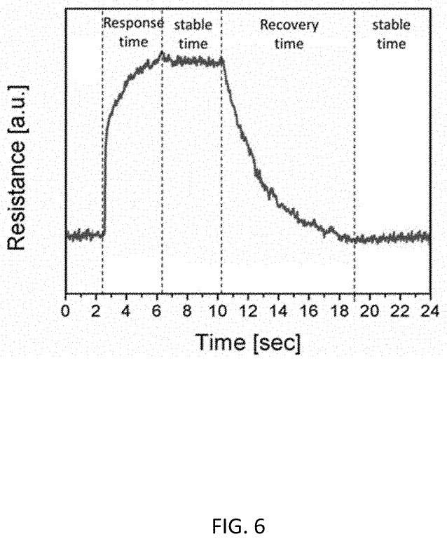

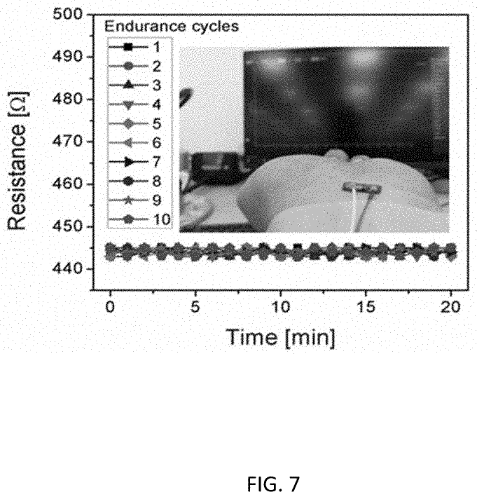

FIGS. 6 and 7 are graphs showing experimental results of a resistance test of a wearable sensor on a wrist of a user. For this analysis, an example wearable sensor according to the present disclosure was connected to a source meter (e.g., detecting/processing circuit) through leads and was placed on the wrist with the help of plastic tape. As shown in these figures, initially the wearable sensor showed high resistance and as the wearable sensor was detached from the wrist, its resistance decreased gradually. After the recovery time, when the wearable sensor was attached again on the wrist, the resistance increased again.

When the wearable sensor is worn on a wrist of a user, it may cause a change in temperature surrounding the wearable sensor. As shown in FIG. 6, the wearable sensor showed a response time of around 4 seconds, which is the time that took for the resistance to transition from a low value to a high value (on the time scale from 2.2 to 6.2 seconds) after there was a change in temperature surrounding the wearable sensor. After the 4 seconds duration, the resistance of the wearable sensor became stable (e.g., between 6.2 to 10 seconds). The resistance value in this stable state may correspond to the changed temperature (e.g., body temperature of the user). When the wearable sensor was removed from the wrist, the time that took for the resistance to transition from the high value to the low value was recorded. As shown in FIG. 6, the recovery time was around 8.5 seconds.

The response time and recovery time can be tuned by controlling various parameters of the components of the wearable sensor (e.g., thickness of the substrate/encapsulation layer, properties of the sensing film/substrate, etc.). In some examples, the wearable sensor according to the present disclosure may have a response time in a range of about 3 seconds to about 10 seconds_{[A2]}. In other examples, the wearable sensor may have any other suitable response time. In some examples, the wearable sensor according to the present disclosure may have a recovery time in a range of about 5 seconds to about 15 seconds. In other examples, the wearable sensor may have any other suitable recovery time.

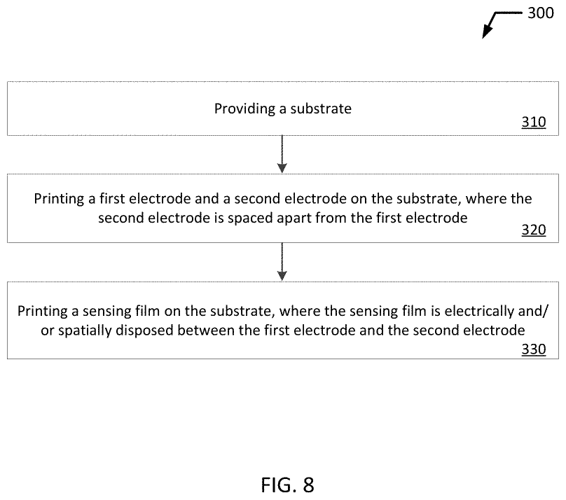

FIG. 8 is a flowchart illustrating an example method 300 for manufacturing a wearable sensor according to an example embodiment of the present disclosure. Although the example method 300 is described with reference to the flowchart illustrated in FIG. 8, it will be appreciated that many other methods of performing the acts associated with the method may be used. For example, the order of some of the blocks may be changed, certain blocks may be combined with other blocks, and some of the blocks described are optional.

In the illustrated example, a substrate may be provided (block 310). Then, a first electrode and a second electrode may be printed on the substrate, where the second electrode is spaced apart from the first electrode (block 320). Then, a sensing film may be printed on the substrate, where the sensing film is electrically and/or spatially disposed between the first electrode and the second electrode (block 330). A resistance between the first electrode and the second electrode may change in response to a change in temperature surrounding the sensing film.

In some examples, the method 300 may further include printing an encapsulation layer on the sensing film, the first electrode, and the second electrode to protect the sensing film, the first electrode, and the second electrode from an environmental factor. In some examples, at least one of the first electrode, the second electrode, the sensing film, and the encapsulation layer may be printed using a screen-printing technique or a 3D printing technique. In other examples, the first electrode, the second electrode, the sensing film, and/or the encapsulation layer may be printed using any other suitable process.

Aspects of the present disclosure may simplify the wearable sensor design as well as the manufacturing process, thereby allowing a mass production of a wearable sensor at a very low cost, and making it easy to change the design according to the target applications.

Human body temperature is a very essential biomarker for the early detection of various diseases. The use of human body temperature is not limited to the clinics, critical patients, and infants, but it can also be utilized in various applications such as sports, outdoor activity, and construction workers. Aspects of the present disclosure may help in rapid and early detection of the body temperature, and various biomarkers, thereby reducing life-threatening incidents.

As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

Reference throughout the specification to “various aspects,” “some aspects,” “some examples,” “other examples,” “some cases,” or “one aspect” means that a particular feature, structure, or characteristic described in connection with the aspect is included in at least one example. Thus, appearances of the phrases “in various aspects,” “in some aspects,” “certain embodiments,” “some examples,” “other examples,” “certain other embodiments,” “some cases,” or “in one aspect” in places throughout the specification are not necessarily all referring to the same aspect. Furthermore, the particular features, structures, or characteristics illustrated or described in connection with one example may be combined, in whole or in part, with features, structures, or characteristics of one or more other aspects without limitation.

When the position relation between two parts is described using the terms such as “on,” “above,” “below,” “under,” and “next,” one or more parts may be positioned between the two parts unless the terms are used with the term “immediately” or “directly.” Similarly, as used herein, the terms “attachable,” “attached,” “connectable,” “connected,” or any similar terms may include directly or indirectly attachable, directly or indirectly attached, directly or indirectly connectable, and directly or indirectly connected.

It is to be understood that at least some of the figures and descriptions herein have been simplified to illustrate elements that are relevant for a clear understanding of the disclosure, while eliminating, for purposes of clarity, other elements. Those of ordinary skill in the art will recognize, however, that these and other elements may be desirable. However, because such elements are well known in the art, and because they do not facilitate a better understanding of the disclosure, a discussion of such elements is not provided herein.

The terminology used herein is intended to describe particular embodiments only and is not intended to be limiting of the present disclosure. As used herein, the singular forms “a,” “an,” and “the” are intended to include the plural forms as well, unless otherwise indicated. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “at least one of X or Y” or “at least one of X and Y” should be interpreted as X, or Y, or X and Y.

It should be understood that various changes and modifications to the examples described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A wearable sensor for real-time human body temperature measurement, the wearable sensor comprising:
a substrate;
a first electrode on the substrate;
a second electrode on the substrate, wherein the second electrode is spaced apart from the first electrode; and
a sensing film on the substrate, wherein the sensing film is electrically and/or spatially disposed between the first electrode and the second electrode,
wherein a resistance between the first electrode and the second electrode changes in response to a change in temperature surrounding the sensing film.

2. The wearable sensor of claim 1, further comprising: an encapsulation layer on the sensing film.

3. The wearable sensor of claim 2, wherein the encapsulation layer covers the first electrode, the second electrode, and the sensing film to protect the first electrode, the second electrode, and the sensing film from an environmental factor.

4. The wearable sensor of claim 3, wherein the environmental factor comprises at least one of humidity and oxidation.

5. The wearable sensor of claim 2, wherein the encapsulation layer comprises polydimethylsiloxane.

6. The wearable sensor of claim 2, wherein the encapsulation layer is waterproof.

7. The wearable sensor of claim 1, wherein a detection temperature range of the wearable sensor is in a range of about 28° C. to about 50° C.

8. The wearable sensor of claim 1, wherein the sensing film comprises carbon black.

9. The wearable sensor of claim 1, wherein the first electrode and the second electrode comprise silver nano-particles.

10. The wearable sensor of claim 1, wherein the first electrode and the second electrode comprise an interdigital electrode having a comb-shaped arrangement.

11. The wearable sensor of claim 1, further comprising a detection/processing circuit configured to process the change of the resistance.

12. The wearable sensor of claim 11, wherein the detection/processing circuit is configured to determine a human body temperature based on the change of the resistance.

13. The wearable sensor of claim 11, wherein the detection/processing circuit is configured to determine a respiration rate based on the change of the resistance.

14. The wearable sensor of claim 1, wherein the change of the resistance is reversible.

15. A method of manufacturing a wearable sensor for real-time human body temperature measurement, the method comprising:
providing a substrate;
printing a first electrode and a second electrode on the substrate, wherein the second electrode is spaced apart from the first electrode; and
printing a sensing film on the substrate, wherein the sensing film is electrically and/or spatially disposed between the first electrode and the second electrode,
wherein a resistance between the first electrode and the second electrode changes in response to a change in temperature surrounding the sensing film.

16. The method of claim 15, further comprising printing an encapsulation layer on the sensing film, the first electrode, and the second electrode to protect the sensing film, the first electrode, and the second electrode from an environmental factor.

17. The method of claim 16, wherein the encapsulation layer comprises polydimethylsiloxane.

18. The method of claim 15, wherein the sensing film comprises carbon black.

19. The method of claim 15, wherein the first electrode and the second electrode comprise silver nano-particles.

20. The method of claim 15, wherein at least one of the first electrode, the second electrode, and the sensing film is printed using a screen-printing technique or a 3D printing technique.
